(19) United States
(12) Patent Application Publication
(10) Pub. No.: US 2012/0171190 A1
(43) Pub. Date: Jul. 5, 2012

(54) METHODS FOR DIAGNOSING CHRONIC DIARRHEA THROUGH PROTEIN SECRETION ANALYSIS

(76) Inventor: Thomas M. Donndelinger, Nampa, ID (US)

(21) Appl. No.: 13/414,594

(22) Filed: Mar. 7, 2012

Related U.S. Application Data
(63) Continuation-in-part of application No. 12/504,580, filed on Jul. 16, 2009.
Application No. 12/504,580 is a continuation-in-part of application No. 11/876,590, filed on Oct. 22, 2007, now abandoned.
Application No. 11/876,590 is a continuation-in-part of application No. 11/613,717, filed on Dec. 20, 2006, now abandoned.
(60) Provisional application No. 60/752,618, filed on Dec. 21, 2005.

(51) Int. Cl. A61K 38/45; G01N 33/566; C12Q 1/68; G01N 33/68; A61P 1/12; C12Q 1/40; C12Q 1/37; C12Q 1/44; A61K 38/17; A61K 38/57; G01N 33/573; C12Q 1/48

(52) U.S. Cl. 424/94.5; 435/7.4; 435/7.1; 436/501; 435/6.12; 435/7.92; 436/86; 435/15; 435/22; 435/24; 435/19; 514/21.92; 514/20.3

(57) ABSTRACT

Methods for diagnosing chronic diarrhea and other gastrointestinal conditions. In the methods, a sample of gastrointestinal secretions is obtained from a control group; or a group who has been diagnosed with either healthy gastrointestinal tracts or with a gastrointestinal condition, like chronic diarrhea. The control group samples are analyzed in any suitable manner to determine the levels of gastrointestinal secretions, including one or more autophagy-related proteins, cytokeratins, digestive enzymes, or other proteins. The results of the sample analysis are used to create a database containing profiles of normal and abnormal gastrointestinal secretions. As the database is created and specific secretion level abnormalities are identified, patients may be diagnosed with these abnormalities and be treated by adjusting the levels of specific secretions. Gastrointestinal samples from subsequent patients may be analyzed and compared with the database to determine which of the patients' secretion levels, if any, are abnormal.

### CROSS-REFERENCE TO RELATED APPLICATIONS

This is a continuation-in-part application of U.S. patent application Ser. No. 12/504,580, filed Jul. 16, 2009, which is a continuation-in-part application of U.S. patent application Ser. No. 11/876,590, filed Oct. 22, 2007, which is a continuation-in-part application of U.S. Utility patent application Ser. No. 11/613,717, filed Dec. 20, 2006, which claims priority to U.S. Provisional Patent Application 60/752,618, filed Dec. 21, 2005, the entire disclosures of which are hereby incorporated by reference.

### BACKGROUND

1. The Field of the Invention

The present disclosure relates to the field of medical diagnosis. More particularly, the present disclosure discusses improved methods for diagnosing or obtaining information about chronic diarrhea and other gastrointestinal disorders in humans or other warm-blooded animals.

2. The Prior State of the Art

Many people suffer from the medical condition of chronic diarrhea, or diarrhea that lasts for more than two weeks. Although persistent, this problem is usually painless and is generally not accompanied by more serious conditions, such as bleeding, anemia, weight loss, or fatigue. Nevertheless, this condition can be a nuisance and an embarrassment to many people. Accordingly, doctors and researchers have long searched for causes of the ailment as well as its treatments.

There are many different causes of chronic diarrhea. For example, some types of chronic diarrhea may be caused by infection, which, in turn, may have been caused by parasites, bacteria, viruses, or the like. In other cases, chronic diarrhea may be caused by medications, antibiotics, food additives, or drugs ingested into the body. In yet other cases, chronic diarrhea may be caused by more serious medical conditions, such as cancer, pre-malignant and malignant lesions, tumors, diabetes, thyroid and other endocrine diseases, food allergies, reduced blood flow to the intestine, colitis, and/or Crohn's disease. Previous surgery or radiation treatments on the abdomen or gastrointestinal tract may also be the source of some forms of chronic diarrhea.

Yet, despite the knowledge regarding chronic diarrhea, there are some cases of chronic diarrhea for which there are no known causes. In fact, it is estimated that 20% to 30% of all cases of chronic diarrhea have no known cause. Despite numerous tests and analyses, doctors often cannot pinpoint why some patients suffer from chronic diarrhea.

In some cases, doctors may advise patients that the chronic diarrhea problem is caused by stress, emotional problems, psychological problems, and so forth. Accordingly, these doctors may suggest that patients change their lifestyle—by reducing stress, seeking professional counseling, etc.—as a means of coping with the chronic diarrhea. Of course, such treatments can be difficult to implement, expensive, drastic, and ineffective. Thus, such treatments are often disfavored.

Moreover, many of the tests currently used to diagnose chronic diarrhea may require multiple analyses by a doctor. Not only can such a battery of tests require a patient to repeatedly visit the doctor, but more importantly, some of the tests required to diagnose chronic diarrhea may be unreliable as well as uncomfortable, embarrassing, and unpleasant.

Accordingly, it would be an advancement in the art to provide a new method for diagnosing gastrointestinal disorders, which may be easily administered and reliable. Moreover, this new method may also provide doctors and researchers with additional information regarding the cause of the chronic diarrhea. Such a method for diagnosing chronic diarrhea is disclosed herein.

### BRIEF SUMMARY

The present invention is directed to methods for diagnosing or obtaining information about chronic diarrhea in humans or any other desired warm-blooded animals. The invention is based upon a non-binding theory that through one or more biological mechanisms, a healthy gastrointestinal tract secretes a variety of diverse proteinaceous substances into the intestines, and more specifically, into the colon. Some non-limiting examples of such proteinaceous excretions include one or more autophagy-related proteins, cytokeratin I proteins, cytokeratin II proteins, digestive enzymes, antimicrobial proteins, cytoplasm, and/or mucous.

Regarding autophagy-related proteins, it is theorized that one or more autophagy-related proteins (such as BRSK1, BRSK2, CAMK1, CAMK1D, CAMK1G, CAMK2A, CAMK2B, CAMK2D, CAMK2G, DAPK3, DCLK1, and a variety of other proteins that trigger, are part of a protein cascade in, or that are otherwise related to an autophagic process in the gastrointestinal tract) help in an autophagic process that breaks down, catabolizes, and recycles aged, dead, diseased, or damaged cells from the epithelium of the gastrointestinal tract. As part of this theory, it is believed that a proper level of autophagy-related proteins (and hence normal levels of autophagocytosis) in the gastrointestinal tract's epithelial lining may help keep the tract healthy and functioning properly. For instance, such proteins may help keep the gastrointestinal tract healthy by exposing the immune system to (and allowing the immune system to mount a defense against) antigens (such as HIV, colonic epithelial cell antigens, etc.) found on or in cells that are being autophagocytosed. It is further believed that abnormal levels of one or more autophagy-related proteins (and hence abnormal levels of autophagocytosis) in the gastrointestinal tract may lead to, or be an indication of, gastrointestinal disease, such as chronic diarrhea.

With respect to cytokeratin I and cytokeratin II secretions, it is theorized that various cytokeratin I and cytokeratin II subtype proteins or subtypes (such as CK1, CK2, CK5, CK6, CK9, CK10, CK14, and CK16) that are secreted into the gastrointestinal tract may act to stabilize mucous and improve the mucous' ability to attach to cells lining the tract. Accordingly, in this theory, it is believed that the presence of proper levels of certain cytokeratin I and cytokeratin II subtype secretions may help to maintain a healthy protective layer of mucous, which, in turn, controls bacterial antigen access to the cell lining of the gastrointestinal tract. As mentioned above, it is also theorized that the presence of digestive enzymes may indicate a colonic extension or supplementation to normal digestion.

Regarding the role of anti-microbial proteins in the gastrointestinal tract, it is theorized that proteins, such as lysozyme, calprotectin, and other polycationic antimicrobials, like histones, may work together or independently to help regulate intestinal flora. For instance, it is believed that lysozyme, calprotectin, and/or histones may have antibacterial characteristics that help purge the intestines of unhealthy microorganisms.

Under the non-binding theories proposed herein, chronic diarrhea and other gastrointestinal disorders, including cancer or disease in the gastrointestinal tract, may be diagnosed by detecting relatively high, relatively low, or otherwise abnormal levels of one or more of the aforementioned secretions of the gastrointestinal tract.

Accordingly, the described methods may include obtaining a sample of gastrointestinal secretion from a patient. The sample may then be analyzed using one or more conventional or novel techniques. The results of the analysis may then be compared with results obtained from the analysis of control samples, or from samples obtained from individuals who have been diagnosed as having healthy intestines, chronic diarrhea, and/or any other condition or disorder of the gastrointestinal tract. Thus, the control samples may comprise normal and abnormal profiles. Also, if appropriate, the abnormal control samples may be further subset into subcategories of chronic diarrhea or other gastrointestinal disorders.

After the patient's results have been compared with the results of the control samples, any abnormal secretion levels in the patient may be determined. One or more abnormal secretion levels may then be correlated with chronic diarrhea or some other form of gastrointestinal disorder. Additionally, the results of the sample obtained from the patient may also be used to provide more diagnostic information about the type or cause of the condition.

After diagnosis, the patient may be treated for the disorder through regulation or adjustment of the abnormal secretion level. One non-limiting example of such a treatment includes providing a patient with preparations that will block or reduce the production of a protein that is being excessively secreted in a diseased colon. In another example, however, such a treatment includes providing the patient with a supplement that increases the level of one or more proteins that have been determined to be deficient in the patient's gastrointestinal tract.

### BRIEF DESCRIPTION OF THE SEVERAL VIEWS OF THE DRAWINGS

FIG. 1 depicts a side, cross-sectional view of a portion of a small intestine, showing a conventional theory regarding the fate of some cells produced in intestinal crypts;

FIG. 2 depicts a side, cross-sectional view of a lining of a colon, depicting a non-binding theory of cell fate in an intestinal lining;

FIGS. 3-5 each show a photograph showing a tissue sample that has been stained through the use of an APG7 antibody and an immunoperoxidase stain, and which show one or more cells migrating through a basement cell membrane as part of an autophagic process;

FIG. 6 shows a photograph showing a tissue sample that has been stained through the use of a hematoxylin and eosin stain, and which shows a cell migrating through the basement cell membrane as part of an autophagic process;

FIG. 7 the presence of lysozyme in human colonic lumen, wherein the presence of lysozyme is verified through the use of a lysozyme-specific antibody and an immunoperoxidase stain;

FIG. 8 is a photograph showing the presence of lysozyme in human colonic lumen, wherein the presence of lysozyme is verified through the use of a lysozyme-specific antibody and an immunofluorescent stain;

FIG. 9 is a photograph showing the presence of amylase in human colonic lumen, wherein the presence of amylase is verified through the use of an amylase-specific antibody and an immunoperoxidase stain; and

FIG. 10 is a photograph showing the presence of amylase in human colonic lumen, wherein the presence of amylase is verified through the use of an amylase-specific antibody and an immunofluorescent stain.

Together with the following description, the Figures may help demonstrate and explain the principles of the described methods.

### DETAILED DESCRIPTION

The following description supplies specific details in order to provide a thorough understanding. Nevertheless, the skilled artisan will understand that the described methods and any associated techniques can be implemented and used without employing these specific details. Indeed, the described methods can be placed into practice with any modification and can be used in conjunction with any suitable apparatus, systems, components, and/or techniques conventionally used in the industry. For example, while the description below focuses on diagnosing chronic diarrhea in humans, it may also be implemented for any other warm-blooded animal or may be used to diagnose many other gastrointestinal disorders.

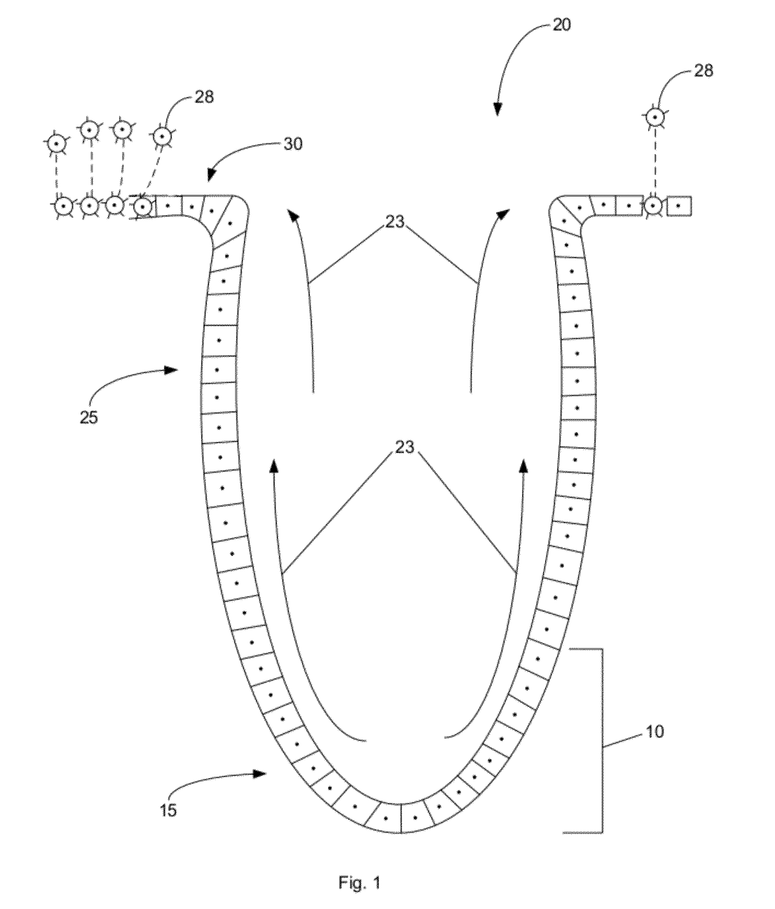

FIG. 1 shows that it is generally understood that stem cells 10 in the intestinal crypts 15 of the small intestine or colon 20 divide. During each cycle of mitosis, it is generally believed that one of the two daughter cells remains in the crypt as a stem cell, while the other cell differentiates and migrates up the side of the crypt (as illustrated by the arrows 23). FIG. 1 further shows that, under some conventional theories, as the differentiated cells 25 die, become sick, age, or otherwise cease to function properly (as shown by cells 28), such cells are sloughed off from the lining 30 of the lumen of the intestines so that they can be passed (along with excrement) from the body.

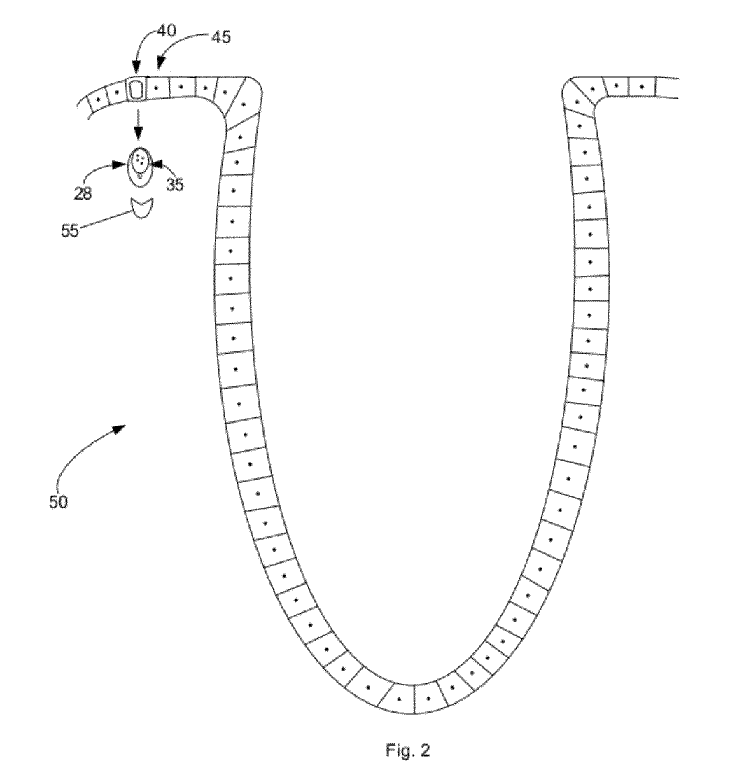

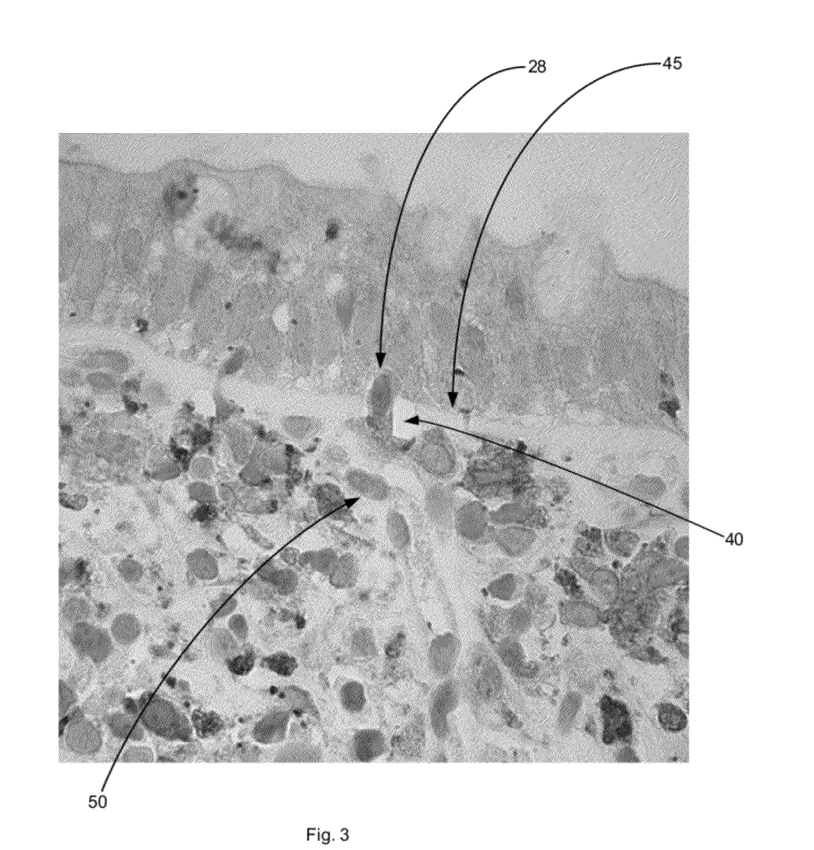

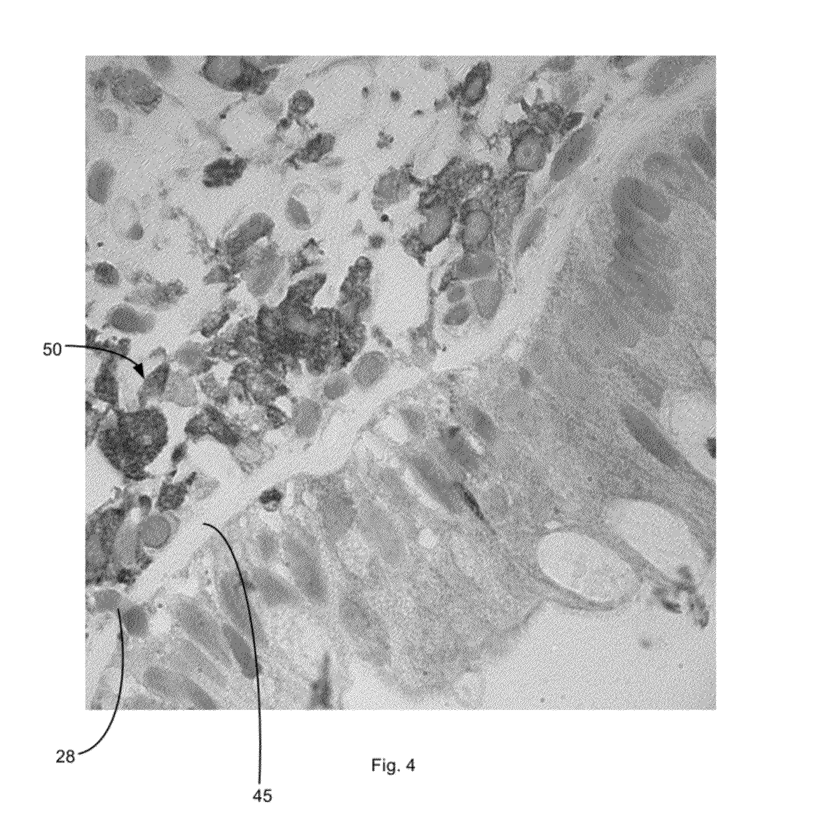

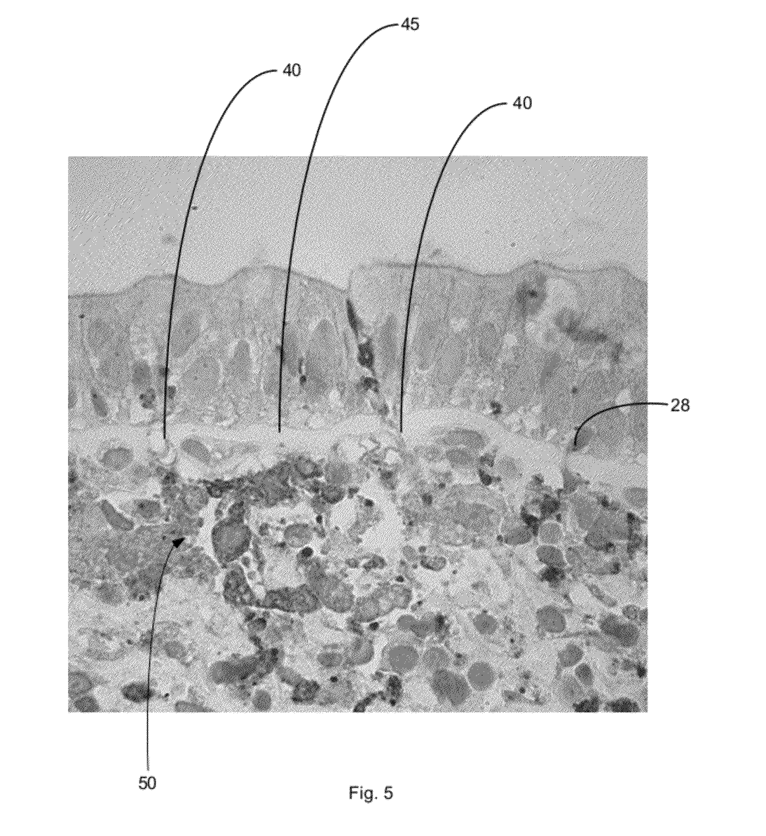

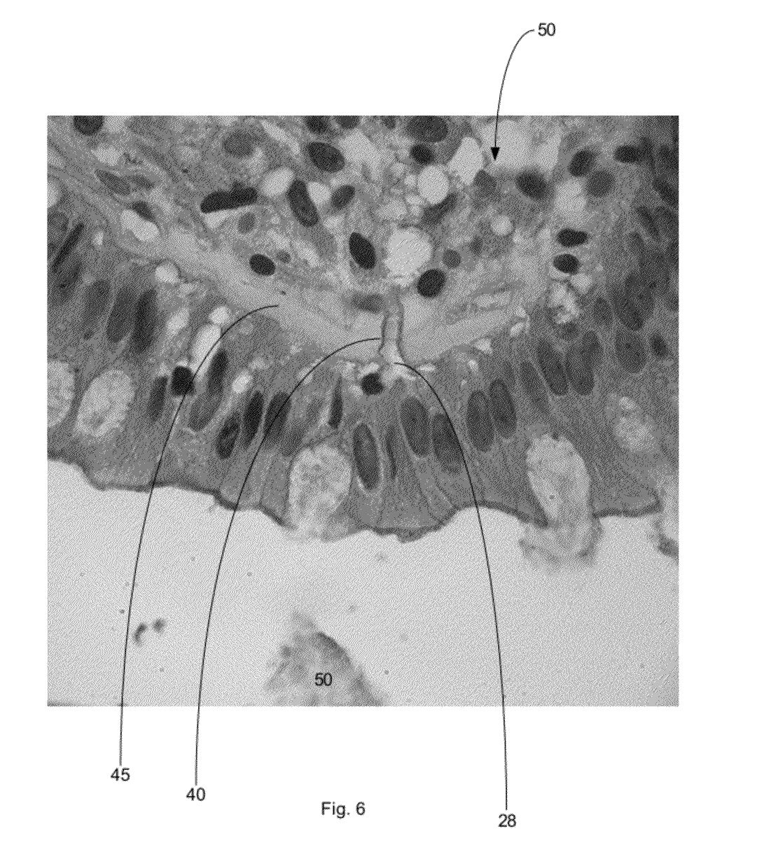

In contrast with the described conventional theory regarding the fate of cells that are produced in the intestinal crypts, Applicant has discovered that instead of being sloughed off from the lining of the intestinal tract, some of the cells produced in the crypts, if not the majority, are signaled to enter autophagocytosis. In this regard, FIG. 2 shows that it is believed that once a cell is signaled to enter into an autophagic process, an autophagic vacuole 35 forms around all or a part of the cell 28. It is further believed that the autophagic vacuole 35 passes through a pore 40 in the basement membrane 45 of the intestines, into the lamina propria 50. As part of this process of autophagocytosis, the autophagic vacuole may combine with a lysosome (not shown) in order to digest and recycle the various components of the cell. Additionally, as the autophagic vacuole enters the lamina propria, the vacuole can be engulfed and digested by a macrophage 55 and, in some cases, can be taken to the spleen for further processing. In support of this theory regarding the fate of cells that are produced in the intestinal crypts, FIGS. 3-6 each show a light microscopy photograph, depicting one or more cells 28 passing through a pore 40 in the basement membrane 45 as part of an autophagic process. Thus, instead of being lost in accordance with the previously-described conventional theory, it is believed that the materials of cells in the gastrointestinal tract that are no longer functioning properly can be broken down (e.g., through an autophagic process) into amino acids, peptides, and other materials that can be recycled for use in other cells.

As part of the described autophagic process, several proteins that trigger, are part of a cascade, are an indication of, or that otherwise participate in autophagocytosis (which proteins may be collectively and individually referred to herein as autophagy-related proteins) are secreted into the gastrointestinal tract. In addition to autophagy-related protein secretions, Applicant has also discovered that humans and other life forms secrete cytokeratin I subtypes, cytokeratin II subtypes, digestive enzymes, antimicrobial proteins, and/or other proteins into the gastrointestinal tract.

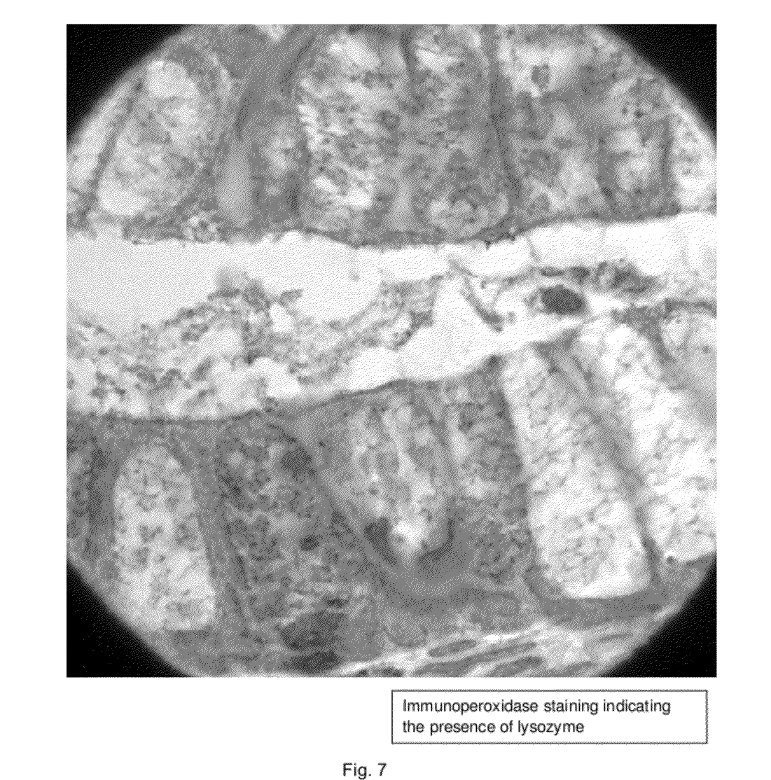

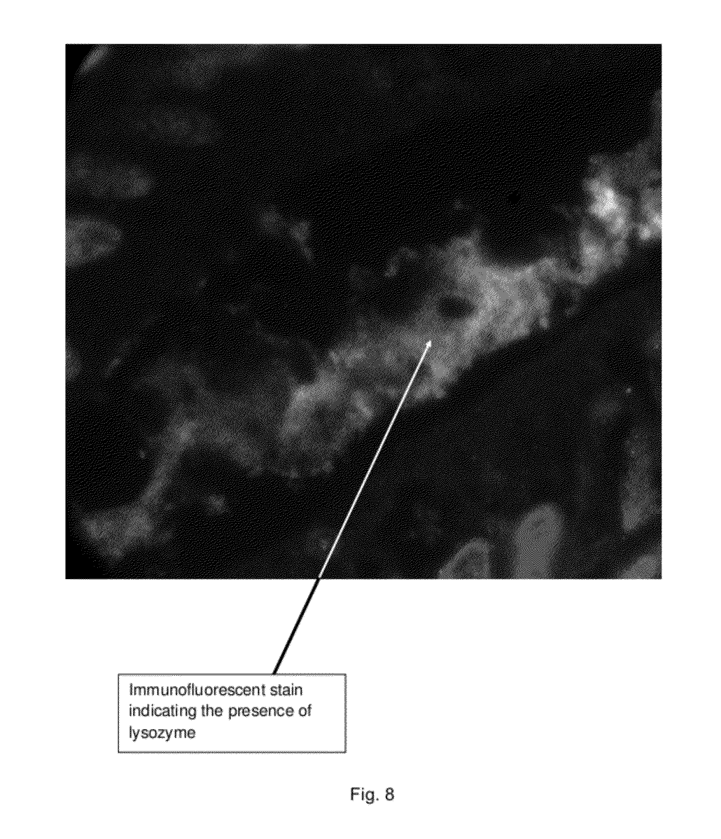

The described gastrointestinal secretions may occur in various places throughout the digestive tract and, in particular, may be found in the colon. For example, evidence that antimicrobial proteins, which may include lysozyme, are secreted into the gastrointestinal tract is shown in FIGS. 7 through 8. FIGS. 7 through 8 contain light microscopy pictures taken at 1,600× of samples that were prepared using the methods disclosed in U.S. Patent Application Publication No. 2006-0228772-A1, entitled “Compositions and Methods for Preparing Specimens for Microscopic Analysis,” the entire disclosure of which is hereby incorporated by reference.

Specifically, FIGS. 7 and 8 are photographs that include arrows indicating the presence of lysozyme in human colonic lumen. While the samples in both FIGS. 7 and 8 have been treated with a rabbit IgG lysozyme primary antibody, the sample in FIG. 7 was treated with an immunoperoxidase secondary antibody while the sample in FIG. 8 was treated with an immunofluorescent secondary antibody.

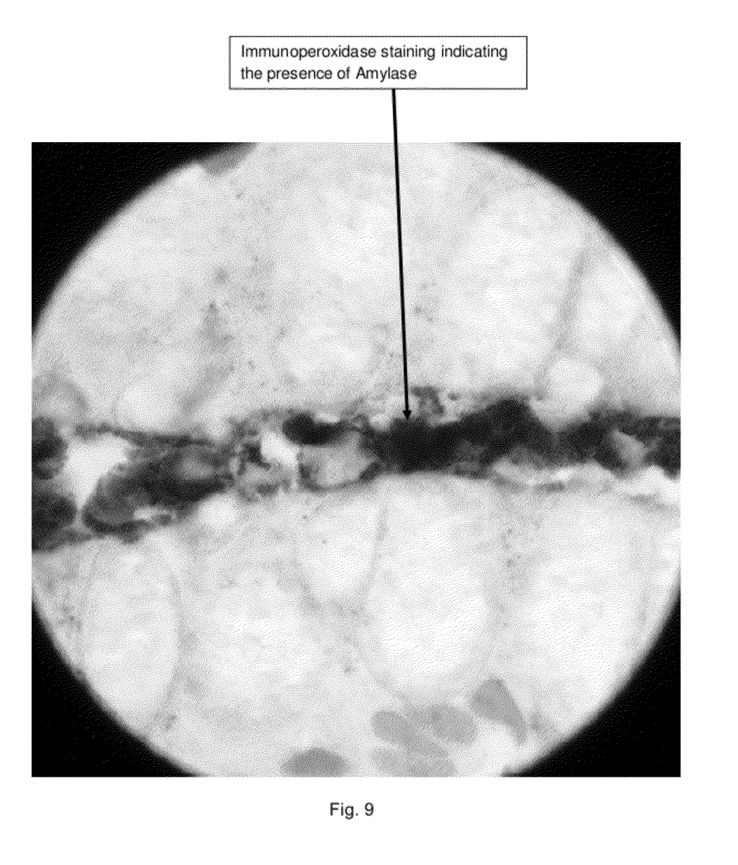

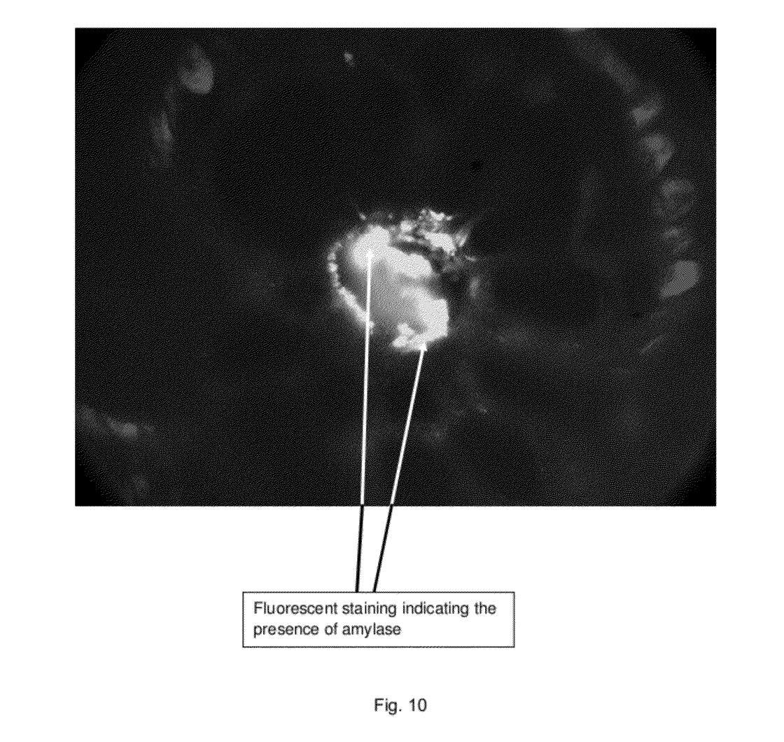

Similarly, evidence that digestive enzyme secretions occur in the gastrointestinal tract may be found in FIGS. 9 and 10, which are also pictures taken using light microscopy according to the aforementioned methods. In particular, FIGS. 9 and 10 show photographs of human colonic lumen that have been treated with a mouse immunoglobulin (IgG) amylase primary antibody, wherein the presence of amylase is indicated with arrows. While the sample in FIG. 9 has been treated with an immunoperoxidase secondary antibody, the sample in FIG. 10 has been treated with an immunofluorescent secondary antibody.

The theory that autophagy-related proteins, cytokeratin I subtypes, cytokeratin II subtypes, digestive enzymes, antimicrobial proteins, and/or mucus are secreted and play a role in maintaining the health of the gastrointestinal tract provides a basis for the described methods. Although the present methods may involve a variety of steps or processes in any suitable order, a non-limiting example of a typical method is described herein for purpose of illustration. Generally, the described method may comprise collecting samples of gastrointestinal secretions from a control group; or an initial group, or groups, of patients (or humans) who have been diagnosed with healthy gastrointestinal tracts, chronic diarrhea, and/or any other desired form of gastrointestinal disorder. The samples from the control group may be analyzed in order to determine specific secretion levels and may be used to create a database containing profiles of normal and abnormal gastrointestinal secretions as well as any subset thereof. After such a database is created and specific secretion level abnormalities are identified, patients with these abnormalities may be treated by regulating or adjusting the specific secretion levels. Furthermore, gastrointestinal samples from subsequent patients may be analyzed and compared with the database in order to determine which of the patients' secretion levels, if any, are abnormal. Once a specific secretion level abnormality has been identified in a patient, the patient may be treated. In order to provide a better understanding of the aforementioned method, this method is discussed below in more detail.

As mentioned, the described method may include collecting samples of gastrointestinal secretions from at least one control group and/or from subsequent patients. The control group in the described methods may be made up of any number of patients with any desired characteristic. For instance, the number of patients in a control group may range anywhere from one single patient to groups of millions of patients, or any number in between. Also, patients in the control group may be of any desired age, sex, weight, height, race, ethnicity, socioeconomic class, profession, and have any other demographic characteristic. Moreover, the patients in the control group may have any known or unknown physical, mental, and/or emotional condition or disorder. For example, patients of the control group may have been previously diagnosed, by any suitable method, as having healthy bowels, chronic diarrhea, and/or another gastrointestinal condition. Some additional non-limiting examples of gastrointestinal conditions may include Crohn's disease, ulcerative colitis, ulcers, cancer, pre-malignant lesions, malignant lesions, etc. Patients in the control group who have been diagnosed with a gastrointestinal disorder may also be subset in any desired manner. For example, patients with chronic diarrhea may be subset into categories or subcategories according to the cause of the diarrhea (e.g., diarrhea caused by disease, parasites, bacteria, viruses, stress, drugs, cancer, specific abnormal secretion levels, surgery, unknown factors, etc.).

In some embodiments, a sample may be collected from a patient's colon (i.e., ascending colon, transverse colon, descending colon, and/or sigmoid colon). In other embodiments, a sample of gastrointestinal secretions may be collected from another portion of a patient's gastrointestinal tract, which may range from a patient's mouth to a patient's anus. In one example, samples are collected from a patient's small intestine or stomach.

The collected samples may contain any secretion that can be found in the gastrointestinal tract. For instance, the gastrointestinal samples may contain one or more autophagy-related proteins, cytokeratin I subtypes, cytokeratin II subtypes, antimicrobial proteins, cytoplasm, mucous, digestive enzymes, and/or markers for cancer. As used herein, the terms secretion, autophagy-related proteins, cytokeratin I subtypes, cytokeratin II subtypes, antimicrobial proteins, digestive enzymes, markers for cancer, and subclasses thereof (e.g., specific proteins) may include gene-spliced variants, post-translational modifications, degradation products, truncations, precursors (e.g., RNA), precursors in a cascade, or other detectable chemical structures that can be used to determine levels of the proteinaceous secretions.

With regards to autophagy-related proteins, samples can contain any proteins that: trigger, are part of a cascade in, or that otherwise participate in or are an indication of an autophagic process in the gastrointestinal tract of a warm-blooded animal. Some non-limiting examples of autophagy-related proteins include BRSK1, BRSK2, CAMK1, CAMK1D, CAMK1G, CAMK2A, CAMK2B, CAMK2D, CAMK2G, DAPK3, DCLK1, MARK1, MARK2, MARK3, MARK4, NUAK1, NUAK2, PLK3, PLK4, PNCK, PRKAA1, PRKAA2, QSK, SIK1, SIK2, SNRK, STK33, STK36, ULK1, ULK2, ULK4, AKT1, AKT2, AKT3, ATG13, CHD9, HARBI1, KIAA0652, RPS6 KB1, RPS6 KB2, SGK1, SGK2, SGK3, CALCOCO1, CCDC21, MY018A, PHLDB2, PLCXD2, ROCK1, TMF1, TPR, BECN1, BECN111, CCDC88A, CEP12, CROCC, CTTNBP2, CTTNBP2NL, DNAH9, EPS15, KIF21A, KIF21B, MYH10, MYH11, MYH9, MY05C, NINL, RAB11FIP3, RAB11FIP4, ROCK2, SWAP70, ATG14, ATG14L, C9orf96, CAMK1, CDKL2, HVPS15, MAPK1, MAPK3, MARK1, MARK2, MARK3, MARK4, PHKG1, HIK3R4, PRKAA1, PRKAA2, QSK, SIK1, SIK2, SIK3, TMEM74, ATG5, OSGEP, OSGEP11, ATG7, MOCS3, UBA1, UBA2, UBA3, UBA6, ATG10, CNNM2, CNNM4, ATG12, DES, EEF1A1, EEF1A2, ENSP00000395046, GFAP, INA, LOC100293909, NEFL, NEFM, PRPH, PRPH2, VIM, ACTA1, ACTA2, ACTB, ACTBL2, ACTC1, ACTG1, ACTG2, ATG1611, ATG16L2, DCTN1, EPS1511, FBXW7, ITSN1, KIF5A, LOC653269, P704P, PAFAH1B1, SNRNP40, TM9SF1, TM9SF2, TM9SF3, TM9SF4, WDR5, WDR5B, WDR69, ATG10, ATG3, ATG4A, ATG4B, ATG4C, ATG4D, ATG7, MOCS3, UBA1, UBA2, UBA6, GABARAP, GABARAPL1, GABARAPL2, GABARAPL3, LOC652191, MAP1LC3A, MAP1LC3B, MAP1LC3B2, MAP1LC3C, SSSCA1, ULK3, ATG2A, ATG2B, VPS13A, VPS13C, VPS13D, NLE1, WDR45, WDR45L, WIPI1, WIPI2, ABCB6, APEX1, ATG9A, ATG9B, C11orf24, C20orf151, C2orf16, CIC, FGFR1, FLG, FLG2, FMN2, FNDC1, IWS1, LEO1, MAP7D1, Mb3875, MLL2, MLL4, MUC1, NACA, PCLO, POLR2A, PRB3, REEP6, RERE, SETD5, ZNF828, CUX1, DNAH5, DNAH8, LOC652164, LOC653513, LOC727927, MY018A, PDE4DIP, TIAF1, SNX1, SNX18, SNX2, SNX3, SNX32, SNX5, SNX6, BCAS3, PRAF2, WDR45, WER45L, WIPI1, WIPI2, AZI1, CCDC88C, DCD42BPB, DSP, DSPP, HIP1, KIF16B, LAMA2, SPTBN5, SYNE2, TAX1BP1, UACA, ACTA1, ACTA2, ACTB, ACTC1, ACTG1, ACTG2, ACTR2, ACTR3, ACTR3B, ACTR3C, ENSP00000380341, SNX2, SNX30, SNX33, SNX4, SNX7, IGF2R, CLSTN1, HPVS34, PIK3C2A, PIK3C2B, PIK3C2G, PIK3C3, PIK3CA, PIK3CB, PIK3CD, PIK3CG, CUX1, DNAH5, DNAH8, LOC652164, LOC653513, LOC727927, MY018A, PDE4DIP, TIAF1, LOC100294331, SLC2A1, SLC2A14, SLC2A2, SLC2A3, SLC2A4, SLC2A5, SLC2A7, SLC2A9, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17UGT2B28, UGT2B4, UGT2B7, UGT3A2, UGT8, LOC652797, PKLR, PKM2, FLVCR1, FLVCR2, LOC100133772, SLC16A5, ANTXR1, ATM, ATR, GCN1L1, LOC440354, LOC648152, LOC651610, LOC651921, LOC731751, MMAB, MTOR, PRKDC, SERPINA2, SMG1, TRRAP, ANTXR1, ATM, ATR, LOC100288704, LOC440354, LOC648152, LOC651610, LOC651921, LOC731751, MMAB, PRKDC, SERPINA2, SMG1, TRRAP, DCD42BPA, EZR, UVRAG, WDR87, ARHGAP17, ARHGAP44, SH3GL1, SH3GL2, SH3GL3, SH3GLB1, SH3GLB2, DRAM, DRAM1, DRAM2, TMEM150A, TMEM150B, TMEM150C, AMBRA1, FBXW7, KATNB1, POC1A, POC1B, SPAG16, WDR38, WDR47, WDR5, WDR5B, WDR69, AKAP9, CGNL1, CIT, CLIP1, DHDDS, KIF15, MYH1, MYH13, MYH4, MYH8, RB1CC1, C120RF44, EEA1, FGD4, FGD5, FGD6, HGS, MTMR3, MTMR4, PIKFYVE, PLEKHF2, RUFY1, RUFY2, WDFY2, ZFYVE1, ZFYVE28, BAK1, BAX, BCL2, BCL2A1, BCL2L1, BCL2L2, BOK, LOC100289713, MCL1, DLG1, DLG2, DLG3, DLG4, GOPC, GRIP1, GRIP2, INADL, LGALS12, LIN7A, LIN7B, LIN7C, LLGL1, LNX1, MPDZ, MPP2, MPP3, MYH2, MYH3, MYH6, MYH7, NCOA2, PDZRN3, SNTA1, SNTB1, SNTB2, SNTG1, SNTG2, TMEM49, RAB11A, RAB11B, RAB17, RAB1A, RAB1B, RAB22A, RAB31, RAB43, RAB5A, RAB5B, RAB5C, RAB6A, IL18R1, IL1RAP, MYD88, TIRAP, TLR1, TLR10, TLR2, TLR4, TLR6, RUBICON, P62/SQSTM1, C6orf106, HERC2, MIB1, MIB2, NBR1, SQSSTM1, ZZEF1, BIF-1, VMP1, TP531NP1, TP531NP2, BNIP3, BNIP3L, ARMC4, KPNA1, KPNA2, KPNA3, KPNA4, KPNA5, KPNA6, LC3B, GFAP, EF1A, TM9SF1, TMEM166, TMEM74, ULK3, P27, APE1, ARP2, ARP3, and any suitable combination thereof.

With respect to cytokeratin I and cytokeratin II subtypes, the gastrointestinal samples may contain the low molecular weight, acidic type I cytokeratins and/or the high molecular weight, basic or neutral type II cytokeratins. Further, the gastrointestinal samples may contain any known or novel form of a cytokeratin II subtype, such as CK1, CK2, CK3, CK4, CK5, CK6, CK7, and CK8, and/or any known or novel form of a cytokeratin I subtype, such as CK9, CK10, CK12, CK13, CK14, CK16, CK17, CK18, CK19, and CK20. For instance, as will be discussed below in the Examples section, secretions of CK1, CK2, CK5, CK6, CK9, CK10, CK14, and CK16 have been found in gastrointestinal samples taken from human colons.

With respect to the digestive enzymes that may be found in the gastrointestinal samples, the samples may include any digestive enzymes found in the gastrointestinal tracts of warm-blooded animals. Some non-limiting examples of such digestive enzymes may include any appropriate form of amylase (e.g., α-amylase, β-amylase, and/or γ-amylase), lipase, peptidase, sucrase, maltase, lactase, isomaltase, eripsin, gelatinase, trypsin, chymotrypsin, etc., and/or combinations thereof.

Regarding antimicrobial proteins, the samples may include any antimicrobial proteins that are found in the digestive tract of warm-blooded animals. In this regard, some examples of antimicrobial proteins include, but are not limited to, lysozyme, calprotectin, and other polycationic antimicrobials, like histones.

Samples of gastrointestinal secretions may be collected in any suitable manner. Accordingly, the samples may be collected from stool samples or while the patient is undergoing endoscopy or surgery. For example, samples of gastrointestinal secretions that contain one or more autophagy-related proteins, cytokeratin I subtypes, cytokeratin II subtypes, digestive enzymes, antimicrobial proteins, and/or another proteinaceous secretions may be collected from stool samples or directly from the colon. Indeed, samples of gastrointestinal secretions containing any gastrointestinal secretion may often be obtained as the patient undergoes endoscopy. Because endoscopy is currently one of the more commonly used tests performed on patients with chronic diarrhea, patients may not need to be subjected to additional procedures in order to accomplish the present methods. Furthermore, endoscopy is usually performed on a patient after the patient's bowel and gastrointestinal system have been purged. Accordingly, if gastrointestinal secretions are collected from a patient during endoscopy, these secretions may likely be substantially clean and free of excrement or other impurities. Thus, gastrointestinal secretions collected in this manner may need little purification in order to be used in the present method.

Once a sample of gastrointestinal secretions has been collected, the sample may be analyzed using any known or novel technique in order to determine the presence of one or more forms of specific secretions and/or the levels of the specific secretions present in the sample. For instance, some non-limiting examples of methods by which a sample of gastrointestinal secretions may be analyzed include spectroscopy, chromatography, immunohistochemistry, immunofluorescence, immunoblotting (e.g., Western blotting), enzyme-linked immunosorbant assays (ELISA), various forms of polymerase chain reaction (e.g., PCR, RT-PCR, etc.), microscopy, or any other appropriate form of biochemical analysis. Indeed, persons skilled in the art may appreciate that other known and novel analytical methods may be used to analyze the gastrointestinal secretions.

In one example of a method for analyzing samples of gastrointestinal secretions, the samples are analyzed using spectroscopy and/or chromatography. In some embodiments, a sample is analyzed using mass spectroscopy (MS) or gas chromatography-mass spectroscopy (GC-MS). In other embodiments, gas chromatography-mass spectroscopy mass spectroscopy (GC-MS/MS) is used in tandem. In yet other embodiments, surface-enhanced laser desorption ionization time-of-flight (“SELDI-TOF”) mass spectroscopy, or matrix-assisted laser desorption ionization time-of-flight (“MALDI-TOF”) mass spectroscopy are used to determine the presence and/or levels of particular secretions in a sample.

Where mass spectroscopic analysis is performed, the samples of gastrointestinal secretions may be prepared for analysis through any commonly known method. For instance, some potential methods of sample preparation may be described in an article by David K. Crockett et al., entitled “Identification of Proteins from Formalin-Fixed Paraffin-Embedded Cells by LC-MS/MS,” the entire disclosure of which is herein incorporated by reference. While the Crockett article relates to recovering tissue specimens from paraffin blocks, it is within the level of skill in the art to prepare samples of gastrointestinal secretions for use with mass spectroscopy, GC-MS/MS, and other analytical methods, through many other methods. For instance, before spectroscopic analysis, a sample of gastrointestinal secretions may be separated into its various components by ultracentrifugation.

As is known in the art, conducting a mass spectrographic analysis on a sample may produce results in the form of a mass chromatogram proteomic pattern with peaks that correspond to the various compounds, proteins, bio-molecules, etc. that are found in the sample. More specifically, the peaks may include information about the proteome of the sample of gastrointestinal secretions, including information about autophagy-related proteins; cytokeratin I subtypes; cytokeratin II subtypes; antimicrobial proteins, such as lysozyme or histones; digestive enzymes, such as amylase, lipase, peptidase, etc.; cytoplasm; cell membranes; mucous; cancer markers; and/or other structures or compounds found in the secretions. Thus, the mass spectroscopy results of the gastrointestinal secretions may include a spectrum or “fingerprint” of expressed genetic information relating to the gastrointestinal tract. Additionally, if the components of the secretions were separated first (e.g., through centrifugation), then separate mass spectroscopy analysis may occur, which may provide a specific proteomic pattern for each component.

In a second example of methods for analyzing samples of gastrointestinal secretions, the sample may be analyzed through any suitable form of immunohistochemistry. For example, monoclonal and/or polyclonal antibodies targeted to one or more specific secretions may be applied to the sample and be allowed to complex with corresponding antigens. In this example, a labeled primary antibody could be applied to an antigen (such as a particular autophagy-related protein, a cytokeratin I subtype, a cytokeratin II subtype, lysozyme, a histone, amylase, lipase, a cancer marker, etc.) in a single stage and a labeled secondary antibody may be used to target a species-specific part of the structure of a primary antibody. In some instances, the use of a secondary antibody may be advantageous because multiple secondary antibodies may bind to a primary antibody and thereby amplify the signal.

Although any suitable primary and/or secondary antibody(s) may be used to target a desired secretion, some non-limiting examples of suitable primary antibodies may include antibodies targeted to: an autophagy-related protein (e.g., anti-BRSK1, anti-BRSK2, anti-CAMK1, anti-CAMK1D, anti-CAMK1G, anti-CAMK2A, anti-CAMK2B, anti-CAMK2D, anti-CAMK2G, anti-DAPK3, anti-DCLK1, anti-MARK1, anti-MARK2, anti-MARK3, anti-MARK4, anti-NUAK1, anti-NUAK2, anti-PLK3, anti-PLK4, anti-PNCK, anti-PRKAA1, anti-PRKAA2, anti-QSK, anti-SIK1, anti-SIK2, anti-SNRK, anti-STK33, anti-STK36, anti-ULK1, anti-ULK2, anti-ULK4, anti-AKT1, anti-AKT2, anti-AKT3, anti-ATG13, anti-CHD9, anti-HARBI1, anti-KIAA0652, anti-RPS6 KB1, anti-RPS6 KB2, anti-SGK1, anti-SGK2, anti-SGK3, anti-CALCOCO1, anti-CCDC21, anti-MY018A, anti-PHLDB2, anti-PLCXD2, anti-ROCK1, anti-TMF1, anti-TPR, anti-BECN1, anti-BECN111, anti-CCDC88A, anti-CEP12, anti-CROCC, anti-CTTNBP2, anti-CTTNBP2NL, anti-DNAH9, anti-EPS15, anti-KIF21A, anti-KIF21B, anti-MYH10, anti-MYH11, anti-MYH9, anti-MY05C, anti-NINL, anti-RAB11FIP3, anti-RAB11FIP4, anti-ROCK2, anti-SWAP70, anti-ATG14, anti-ATG14L, anti-C9orf96, anti-CAMK1, anti-CDKL2, anti-HVPS15, anti-MAPK1, anti-MAPK3, anti-MARK1, anti-MARK2, anti-MARK3, anti-MARK4, anti-PHKG1, anti-HIK3R4, anti-PRKAA1, anti-PRKAA2, anti-QSK, anti-SIK1, anti-SIK2, anti-SIK3, anti-TMEM74, anti-ATG5, anti-OSGEP, anti-OSGEP11, anti-ATG7, anti-MOC53, anti-UBA1, anti-UBA2, anti-UBA3, anti-UBA6, anti-ATG10, anti-CNNM2, anti-CNNM4, anti-ATG12, anti-DES, anti-EEF1A1, anti-EEF1A2, anti-ENSP00000395046, anti-GFAP, anti-INA, anti-LOC100293909, anti-NEFL, anti-NEFM, anti-PRPH, anti-PRPH2, anti-VIM, anti-ACTA1, anti-ACTA2, anti-ACTB, anti-ACTBL2, anti-ACTC1, anti-ACTG1, anti-ACTG2, anti-ATG1611, anti-ATG16L2, anti-DCTN1, anti-EPS1511, anti-FBXW7, anti-ITSN1, anti-KIF5A, anti-LOC653269, anti-P704P, anti-PAFAH1B1, anti-SNRNP40, anti-TM9SF1, anti-TM9SF2, anti-TM9SF3, anti-TM9SF4, anti-WDR5, anti-WDR5B, anti-WDR69, anti-ATG10, anti-ATG3, anti-ATG4A, anti-ATG4B, anti-ATG4C, anti-ATG4D, anti-ATG7, anti-MOCS3, anti-UBA1, anti-UBA2, anti-UBA6, anti-GABARAP, anti-GABARAPL1, anti-GABARAPL2, anti-GABARAPL3, anti-LOC652191, anti-MAP1LC3A, anti-MAP1LC3B, anti-MAP1LC3B2, anti-MAP1LC3C, anti-SSSCA1, anti-ULK3, anti-ATG2A, anti-ATG2B, anti-VPS13A, anti-VPS13C, anti-VPS13D, anti-NLE1, anti-WDR45, anti-WDR45L, anti-WIPI1, anti-WIPI2, anti-ABCB6, anti-APEX1, anti-ATG9A, anti-ATG9B, anti-C11orf24, anti-C20orf151, anti-C2orf16, anti-CIC, anti-FGFR1, anti-FLG, anti-FLG2, anti-FMN2, anti-FNDC1, anti-IWS1, anti-LEO1, anti-MAP7D1, anti-Mb3875, anti-MLL2, anti-MLL4, anti-MUC1, anti-NACA, anti-PCLO, anti-POLR2A, anti-PRB3, anti-REEP6, anti-RERE, anti-SETD5, anti-ZNF828, anti-CUX1, anti-DNAH5, anti-DNAH8, anti-LOC652164, anti-LOC653513, anti-LOC727927, anti-MY018A, anti-PDE4DIP, anti-TIAF1, anti-SNX1, anti-SNX18, anti-SNX2, anti-SNX3, anti-SNX32, anti-SNX5, anti-SNX6, anti-BCAS3, anti-PRAF2, anti-WDR45, anti-WER45L, anti-WIPI1, anti-WIPI2, anti-AZI1, anti-CCDC88C, anti-DCD42BPB, anti-DSP, anti-DSPP, anti-HIP1, anti-KIF16B, anti-LAMA2, anti-SPTBN5, anti-SYNE2, anti-TAX1BP1, anti-UACA, anti-ACTA1, anti-ACTA2, anti-ACTB, anti-ACTC1, anti-ACTG1, anti-ACTG2, anti-ACTR2, anti-ACTR3, anti-ACTR3B, anti-ACTR3C, anti-ENSP00000380341, anti-SNX2, anti-SNX30, anti-SNX33, anti-SNX4, anti-SNX7, anti-IGF2R, anti-CLSTN1, anti-HPVS34, anti-PIK3C2A, anti-PIK3C2B, anti-PIK3C2G, anti-PIK3C3, anti-PIK3CA, anti-PIK3CB, anti-PIK3CD, anti-PIK3CG, anti-CUX1, anti-DNAH5, anti-DNAH8, anti-LOC652164, anti-LOC653513, anti-LOC727927, anti-MY018A, anti-PDE4DIP, anti-TIAF1, anti-LOC100294331, anti-SLC2A1, anti-SLC2A14, anti-SLC2A2, anti-SLC2A3, anti-SLC2A4, anti-SLC2A5, anti-SLC2A7, anti-SLC2A9, anti-UGT1A1, anti-UGT1A10, anti-UGT1A3, anti-UGT1A4, anti-UGT1A5, anti-UGT1A6, anti-UGT1A7, anti-UGT1A8, anti-UGT1A9, anti-UGT2A1, anti-UGT2A2, anti-UGT2A3, anti-UGT2B10, anti-UGT2B11, anti-UGT2B15, anti-UGT2B17UGT2B28, anti-UGT2B4, anti-UGT2B7, anti-UGT3A2, anti-UGT8, anti-LOC652797, anti-PKLR, anti-PKM2, anti-FLVCR1, anti-FLVCR2, anti-LOC100133772, anti-SLC16A5, anti-ANTXR1, anti-ATM, anti-ATR, anti-GCN1L1, anti-LOC440354, anti-LOC648152, anti-LOC651610, anti-LOC651921, anti-LOC731751, anti-MMAB, anti-MTOR, anti-PRKDC, anti-SERPINA2, anti-SMG1, anti-TRRAP, anti-ANTXR1, anti-ATM, anti-ATR, anti-LOC100288704, anti-LOC440354, anti-LOC648152, anti-LOC651610, anti-LOC651921, anti-LOC731751, anti-MMAB, anti-PRKDC, anti-SERPINA2, anti-SMG1, anti-TRRAP, anti-DCD42BPA, anti-EZR, anti-UVRAG, anti-WDR87, anti-ARHGAP17, anti-ARHGAP44, anti-SH3GL1, anti-SH3GL2, anti-SH3GL3, anti-SH3GLB1, anti-SH3GLB2, anti-DRAM, anti-DRAM1, anti-DRAM2, anti-TMEM150A, anti-TMEM150B, anti-TMEM150C, anti-AMBRA1, anti-FBXW7, anti-KATNB1, anti-POC1A, anti-POC1B, anti-SPAG16, anti-WDR38, anti-WDR47, anti-WDR5, anti-WDR5B, anti-WDR69, anti-AKAP9, anti-CGNL1, anti-CIT, anti-CLIP1, anti-DHDDS, anti-KIF15, anti-MYH1, anti-MYH13, anti-MYH4, anti-MYH8, anti-RB1CC1, anti-C120RF44, anti-EEA1, anti-FGD4, anti-FGD5, anti-FGD6, anti-HGS, anti-MTMR3, anti-MTMR4, anti-PIKFYVE, anti-PLEKHF2, anti-RUFY1, anti-RUFY2, anti-WDFY2, anti-ZFYVE1, anti-ZFYVE28, anti-BAK1, anti-BAX, anti-BCL2, anti-BCL2A1, anti-BCL2L1, anti-BCL2L2, anti-BOK, anti-LOC100289713, anti-MCL1, anti-DLG1, anti-DLG2, anti-DLG3, anti-DLG4, anti-GOPC, anti-GRIP1, anti-GRIP2, anti-INADL, anti-LGALS12, anti-LIN7A, anti-LIN7B, anti-LIN7C, anti-LLGL1, anti-LNX1, anti-MPDZ, anti-MPP2, anti-MPP3, anti-MYH2, anti-MYH3, anti-MYH6, anti-MYH7, anti-NCOA2, anti-PDZRN3, anti-SNTA1, anti-SNTB1, anti-SNTB2, anti-SNTG1, anti-SNTG2, anti-TMEM49, anti-RAB11A, anti-RAB11B, anti-RAB17, anti-RAB1A, anti-RAB1B, anti-RAB22A, anti-RAB31, anti-RAB43, anti-RAB5A, anti-RAB5B, anti-RAB5C, anti-RAB6A, anti-IL18R1, anti-IL1RAP, anti-MYD88, anti-TIRAP, anti-TLR1, anti-TLR10, anti-TLR2, anti-TLR4, anti-TLR6, anti-RUBICON, anti-P62/SQSTM1, anti-C6orf106, anti-HERC2, anti-MIB1, anti-MIB2, anti-NBR1, anti-SQSSTM1, anti-ZZEF1, anti-BIF-1, anti-VMP1, anti-TP531NP1, anti-TP531NP2, anti-BNIP3, anti-BNIP3L, anti-ARMC4, anti-KPNA1, anti-KPNA2, anti-KPNA3, anti-KPNA4, anti-KPNA5, anti-KPNA6, anti-LC3B, anti-GFAP, anti-EF1A, anti-TM9SF1, anti-TMEM166, anti-TMEM74, anti-ULK3, anti-P27, anti-APE1, anti-ARP2, anti-ARP3, and any suitable combination thereof); cytokeratin (e.g., anti-CK1, anti-CK2, anti-CK3, anti-CK4, anti-CK5, anti-CK6, anti-CK7, anti-CK8, anti-CK9, anti-CK10, anti-CK12, anti-CK13, anti-CK14, anti-CK16, anti-CK17, anti-CK18, anti-CK19, anti-CK20, CK3-11D5, and/or CK3-3E4); a histone protein (e.g., anti-histone H2A, anti-histone H2B, anti-histone H3, anti-histone H4, anti-histone H1, etc.); an antimicrobial protein (e.g., anti-lysozyme); a digestive enzyme (e.g., anti-amylase; anti-lipase; and/or anti-peptidase); or any other suitable protein (e.g., anti-cytochrome oxidase; anti-superoxide dismutase; etc.).

As previously alluded to, whether antibodies are used alone, in a single stage, or in conjunction with other antibodies, in multiple stages, the primary and/or secondary antibodies may be directly conjugated with a label. A label may be any chemical group or radioactive atom added to a molecule that is used in order to track material through a reaction or to locate material spatially. In some embodiments, a color or radioactivity may be emitted from the site of an antibody-antigen complex and the emissions may be of varying intensities according to the level of antigen present.

Where immunohistochemistry is implemented for gastrointestinal secretion analysis, any suitable label may be used to help identify desired secretions and/or to determine secretion levels present in the samples. Some examples of suitable labels may include biotin, Texas red, fluorescein, rhodamine, green florescent protein, red florescent protein, cyan florescent protein, yellow fluorescent protein, horseradish peroxidase, alkaline phosphatase, cyanine, ferratin, phycoerythrin, colloidal gold spheres, gold nano-particles, radiolabels (such as P32, S35, C14, H3, and I125), and the like.

As is known in the art, the results obtained from immunohistochemistry performed on a sample may allow a person or an instrument to analyze the sample and detect the presence of desired forms of secretions and/or the levels of secretions in the sample. The results from such an analysis may be obtained and interpreted in any suitable manner. For example, where the intensity of staining correlates with the amount of protein of interest, a densitometer or absorptiometer may be used to measure protein concentration in a sample. In a second example, a conventional light microscope using bright-field, phase contrast, Nomarski, and/or dark-field microscopy may be used to identify the presence, and to some extent, the level of one or more secretions according the staining of the labels as well as their intensity. For instance, the number of cells comprising a stained or labeled marker may be estimated through the use of a hemocytometer. In a third example, a microscope with a florescent light (i.e., a confocal microscope) may be used to observe the presence, and to some extent the level, of one or more desired secretions, which can be labeled with a florescent marker, such as fluorescein or rhodamine, via one or more antibodies. In a fourth example, one or more secretions marked with a label, such as ferratin or colloidal gold spheres, may be observed and intensity levels may be noticed through the use of an electron microscope (e.g., transmission, scanning, and/or cryoelectron). In yet a fifth example of a method for using immunohistochemistry to analyze a gastrointestinal sample, one or more secretions marked with a radioactive label may be observed and, to some extent, quantified in a sample through the use of a Geiger counter or film that is sensitive to radioactivity.

In a third example of methods for analyzing samples of gastrointestinal secretions, any suitable form of immunoblotting may be used to determine the presence of specific secretions (including detectable forms thereof) as well as the levels of one or more specific secretions in the sample. For instance, secretions from a sample may be denatured and then be separated through sodium dodecyl sulfate poly-acrylamide gel electrophoresis (SDS/PAGE). After the proteins have been separated, they may be moved from within the gel onto a membrane in order to make the proteins accessible to antibody detection. Such a membrane may be made of nitrocellulose, polyvinylidene difluoride (PVDF), or another appropriate material. After being transferred to the membrane, desired proteins may be probed (detected) using antibodies specific to the target protein, as explained above. For instance, a labeled anti-autophagy-related protein antibody targeted to a specific autophagy-related protein (e.g., BRSK1, BRSK2, CAMK1, CAMK1D, CAMK1G, CAMK2A, CAMK2B, CAMK2D, CAMK2G, DAPK3, DCLK1, MARK1, MARK2, MARK3, etc.) may be complexed with the corresponding autophagy-related protein that is found in the gastrointestinal secretion.

After the primary or secondary antibody produces a signal and any unbound proteins have been washed away, results from sample analysis may be obtained. For instance, size approximations may be taken by comparing stained bands of labeled protein to a standardized marker or ladder loaded during electrophoresis and the amount of target protein may also be indexed to a control. In this manner, the presence of a specific secretion, as well as its level in the sample, may be obtained.

No matter the method of sample analysis, the results of various analyzed samples may be compared to each other and may be used to create a database. Such a database may be created in any desired manner. For example, the results from samples of gastrointestinal secretions of patients diagnosed with various forms of chronic diarrhea or other bowel disorders may be grouped and compared with the results of samples obtained from patients who have been diagnosed as having healthy bowels. By way of non-limiting illustration, the proteomic patterns obtained through spectroscopy of the samples obtained from the various groups (e.g., patients with healthy bowels and patients with chronic diarrhea) may be compared and any differences between the group's proteomes may be determined. Similarly, immunohistochemical results, PCR results, chromatography results, and/or results from other biochemical analysis from samples obtained from the various groups may be compared and the different results (e.g., stain presence, staining intensity, protein presence, protein concentration, etc.) may be determined.

By comparing the results from samples obtained from patients with healthy bowels against the results from samples obtained from patients with various gastrointestinal conditions (e.g., chronic diarrhea, cancer, ulcers, etc.), researchers, doctors, technicians, etc. may be able to identify the secretions present in healthy or normal bowels as well as the normal levels of the various secretions. Such a comparison may also allow researchers, doctors, technicians, etc. to identify the absence or presence of certain secretions as well as levels of particular secretions that are associated with various gastrointestinal conditions.

Such a database may be used for any purpose that aids in the diagnosis or study of chronic diarrhea or another gastrointestinal disorder. In one example of the utility of such a database, researchers may observe that healthy colons contain specific autophagy-related proteins and may further identify normal ranges for such proteins in healthy colons. Furthermore, researchers may observe that patients who have been diagnosed with chronic diarrhea have an increased level of one or more specific autophagy-related proteins in the colon, or at least have abnormal levels of one or more autophagy-related proteins (e.g., levels of the specific autophagy-related proteins may be above or below the range of the levels of the corresponding specific autophagy-related proteins found in healthy colons).

In another similar example, the comparison of the results of samples of gastrointestinal secretions from patients with healthy colons to the results of patients diagnosed with chronic diarrhea (or another gastrointestinal malady) may reveal that patients who suffer from chronic diarrhea have one or more combinations of abnormal levels of one or more autophagy-related proteins (e.g., BRSK1, BRSK2, CAMK1, CAMK1D, CAMK1G, CAMK2A, CAMK2B, CAMK2D, CAMK2G, etc.), cytokeratin protein (e.g., a subtype of cytokeratin I and/or II), antimicrobial proteins (e.g., lysozyme, calprotectin, and other polycationic antimicrobials), and/or digestive enzymes (e.g., amylase, lipase, etc.), where abnormal refers to levels that are above or below those typically found in healthy patients. By way of non-limiting illustration, the comparison of levels of autophagy-related proteins measured from patients diagnosed with healthy bowels and patients diagnosed with chronic diarrhea may reveal that a concentration of a specific autophagy-related protein that is 5%, 10%, 20%, 100%, or any other determined percent above or below the normal levels found in patients with healthy bowels is indicative of chronic diarrhea.

In still another non-limiting example of the utility of such a database, a comparison between the secretions from healthy gastrointestinal tracts and gastrointestinal tracts that have been diagnosed with cancer may reveal the presence of known cancer markers, which may indicate a propensity for or the presence of cancer. Moreover, such a comparison may also be used to identify additional cancer markers that have heretofore been unidentified. In sum, the characteristics of bowels with various gastrointestinal disorders may be identified and one or more abnormal profiles may be created.

After a database has been created, samples from subsequent patients may be compared with the database and be used to diagnose gastrointestinal conditions in the subsequent patients. For example, by comparing the proteomic pattern, immunohistochemical staining pattern, or other secretion analysis results from a patient's sample to the database, an instrument, system, processor, and/or individual may be able to identify any abnormalities in the patient's gastrointestinal secretion levels. Also, according to the type of abnormality, the instrument or individual may be able to identify the type or types of gastrointestinal condition the patient has. For instance, an individual may be able to diagnose a patient as having a form of chronic diarrhea associated with an increased level of one or more autophagy-related proteins (e.g., BRSK1, CAMK2D, etc.) in the colon. Thus, by obtaining and analyzing mass spectroscopic proteomic pattern results, or any other form of results, from a particular patient and then comparing the results with those of known profiles in a database, an instrument or individual may be able to diagnose chronic diarrhea and/or another disorder.

Although the comparison of samples may be done in any appropriate manner, adaptive artificial intelligence bioinformatics may be used to read the pathologic states reflected in the proteomic patterns obtained through mass spectroscopy. Bioinformatic pattern recognition tools can recognize complex data from thousands of proteins simultaneously. This information may indicate that the patient has chronic diarrhea or any other gastrointestinal disorder and provide diagnostic information about the type or cause of the condition. Such a method may both speed comparisons as well as increase their accuracy.

As more results are obtained and analyzed, the database may be further subset in any desired manner. Thus, as the database and comparisons reveal more information about gastrointestinal secretions and their roles, the database may also reflect the advanced knowledge and thereby make it easier to diagnose previously un-diagnosable forms of chronic diarrhea and/or other gastrointestinal conditions.

For example, a database may begin simply with a healthy or normal profile and an abnormal or chronic diarrhea profile. As more samples are collected and differences between the samples are identified, each of the categories may be further subset. For instance, the chronic diarrhea category may be split into several subcategories, such as chronic diarrhea caused (or indicated) by: an abnormally high level (or abnormally low level) of one or more autophagy-related proteins, a cytokeratin deficiency, antimicrobial protein deficiencies, digestive enzyme deficiencies, histone deficiencies, cancer, or any other abnormal secretion level. In this example, each of the subcategories could be further subset. For instance, the category of chronic diarrhea caused by an increased level of autophagy-related proteins may be subset into different categories based on the abnormal levels of one or more specific autophagy-related proteins. By way of non-limiting example, the category of chronic diarrhea caused by an abnormally high level of autophagy-related proteins can be subset into multiple subclasses, such as a subclass related to chronic diarrhea caused by an abnormally high level of BRSK1 and a subclass related to chronic diarrhea caused by an abnormally high level of DAPK3. In this example, the subclasses can be further subdivided in any suitable manner. For instance, the subclasses could be further divided to include abnormal levels of a combination of secretions (e.g., a subclass for abnormal levels of a particular autophagy-related protein and one or more particular cytokeratin proteins). This process of subsetting may be repeated many subsequent times, according to any desired criteria.

Moreover, as differences are identified between the gastrointestinal secretions of healthy gastrointestinal tracts and the secretions of gastrointestinal tracts with a disorder, like chronic diarrhea, any effective treatments for such conditions may be developed. For example, after ranges of specific secretion levels in healthy colons have been identified, the levels of the specific secretion levels in patients with unhealthy colons may be regulated or adjusted in any suitable manner so as to control, reduce, treat, and/or eliminate the condition. For instance, a patient who is identified as having a form of chronic diarrhea that is associated with an abnormal level of one or more of the described gastrointestinal secretions may use dietary supplements, pharmaceuticals, injections, intravenous therapies, transdermal patches, delayed release supplements, or suppositories that are capable of restoring, or aiding the restoration, or gastrointestinal secretions to a normal level. In one example, where a patient suffers from an abnormally high level of a particular protein (e.g., an autophagy-related protein) the patient can receive a treatment (e.g., a supplement, pharmaceutical, injection, suppository, etc.) that functions to inactivate, sequester, block, or otherwise reduce the amount of the excessive protein or otherwise reduce its harmful effects in the patient's gastrointestinal tract. In another example, where the described systems determine that the gastrointestinal tract of a patient suffers from a deficiency of a certain protein (e.g., a cytokeratin I and/or cytokeratin II subtype), the patient can receive a treatment (e.g., a supplement, pharmaceutical, injection, IV, etc.) containing the deficient protein (or a precursor to the protein) in order to raise the levels of that particular protein to a suitable level. In yet another example of a method for treating a patient who has been diagnosed with chronic diarrhea, a patient may be prescribed antimicrobials, reducing agents, polycationic compounds, or other supplements alone or with one or more combined in a cocktail, depending on the patient's individual secretion composition. In still another example, when a secretion abnormality is detected in the first stages of the development of colon cancer (e.g., polyps), then one or more proteins of the autophagy system may be blocked as part of the treatment of the colon cancer.

The following examples are given to illustrate an embodiment within the scope of the present invention. These are given by way of example only, and it is understood that the following examples are not comprehensive or exhaustive of the many types of embodiments of the present invention in accordance with the present invention.

### Examples

In one example of forming a database according to one embodiment of the invention, colon samples were taken from two different colons and the protein secretions from the colons were compared through GC-MS. Specifically, 1 μm sections were taken from the colons and 6 μg samples were removed from the sections through laser dissection. The 6 μg samples were then prepared in the same manner and analyzed with GC-MS. This analysis of the first and second samples revealed the results depicted in Tables 1 and 2.

TABLE 1
Experimental Results from the First Colon Sample.

| Type of Proteinaceous Secretion Detected | Percentage of Total Spectra |
|---|---|
| CK1 | 0.95% |
| CK9 | 0.67% |
| CK2 | 0.25% |
| CK10 | 0.33% |
| CK14 | 0.15% |
| CK5 | 0.05% |
| CK6A | 0.06% |
| CK16 | 0.04% |
| Dermicidin Precursor | 0.03% |
| Serum Albumin Precursor | 0.05% |

TABLE 2
Experimental results from second colon sample.

| Type of Proteinaceous Secretion Detected | Percentage of Total Spectra |
|---|---|
| CK1 | 0.64% |
| CK9 | 0.55% |
| CK2 | 0.23% |
| CK10 | 0.28% |
| CK14 | 0.15% |
| CK5 | 0.05% |
| CK6A | 0.06% |
| CK14 | 0.05% |
| Lysozyme Precursor | 0.06% |
| Trypsin Precursor | 0.58% |
| Serum Albumin Precursor | 0.06% |
| Hornerin (Q86YZ3) | 0.06% |
| Filaggrin (P20930) | 0.04% |
| Outer Membrane Protein Precursor—E. coli | 0.03% |
| O157:H7 (P0A911) | |

Specifically, Tables 1 and 2 show that the first and second colon samples contained a variety of proteinaceous secretions, including CK1, CK2, CK5, CK6A, CK9, CK10, CK14, CK16, lysozyme, trypsin, hornerin, serum albumin precursor, filaggrin, and/or the outer membrane protein precursor for e. coli. By correlating any known diagnosis of these colon samples (e.g., whether one colon is healthy and whether one colon suffers from chronic diarrhea) with the obtained GC-MS fingerprints, the described database may be started. This database can then be expanded as additional colon samples having known diagnosis are tested, compared, and the results are entered into the database.

The present invention may be embodied in other specific forms without departing from its methods or other essential characteristics as broadly described herein and claimed hereinafter. The described embodiments and examples are to be considered in all respects only as illustrative, and not as restrictive or limiting. The scope of the invention is, therefore, indicated by the appended claims, rather than by the foregoing description. All changes that come within the meaning and range of equivalency of the claims are to be embraced within their scope.

1. A method for diagnosing a gastrointestinal disorder in a warm-blooded animal comprising:
obtaining a sample of a gastrointestinal secretion from the warm-blooded animal;
analyzing the sample of gastrointestinal secretion to determine whether a level of a autophagy-related protein in the sample is abnormal; and
correlating an abnormal level of the autophagy-related protein in the sample with the gastrointestinal disorder in the warm-blooded animal from which the sample was obtained.

2. The method of claim 1, wherein the autophagy-related protein is selected from BRSK1, BRSK2, CAMK1, CAMK1D, CAMK1G, CAMK2A, CAMK2B, CAMK2D, CAMK2G, DAPK3, DCLK1, MARK1, MARK2, MARK3, MARK4, NUAK1, NUAK2, PLK3, PLK4, PNCK, PRKAA1, PRKAA2, QSK, SIK1, SIK2, SNRK, STK33, STK36, ULK1, ULK2, ULK4, AKT1, AKT2, AKT3, ATG13, CHD9, HARBI1, KIAA0652, RPS6 KB1, RPS6 KB2, SGK1, SGK2, SGK3, CALCOCO1, CCDC21, MY018A, PHLDB2, PLCXD2, ROCK1, TMF1, TPR, BECN1, BECN111, CCDC88A, CEP12, CROCC, CTTNBP2, CTTNBP2NL, DNAH9, EPS15, KIF21A, KIF21B, MYH10, MYH11, MYH9, MY05C, NINL, RAB11FIP3, RAB11FIP4, ROCK2, SWAP70, ATG14, ATG14L, C9orf96, CAMK1, CDKL2, HVPS15, MAPK1, MAPK3, MARK1, MARK2, MARK3, MARK4, PHKG1, HIK3R4, PRKAA1, PRKAA2, QSK, SIK1, SIK2, SIK3, TMEM74, ATG5, OSGEP, OSGEP11, ATG7, MOCS3, UBA1, UBA2, UBA3, UBA6, ATG10, CNNM2, CNNM4, ATG12, DES, EEF1A1, EEF1A2, ENSP00000395046, GFAP, LOC100293909, NEFL, NEFM, PRPH, PRPH2, VIM, ACTA1, ACTA2, ACTB, ACTBL2, ACTC1, ACTG1, ACTG2, ATG1611, ATG16L2, DCTN1, EPS1511, FBXW7, ITSN1, KIF5A, LOC653269, P704P, PAFAH1B1, SNRNP40, TM9SF1, TM9SF2, TM9SF3, TM9SF4, WDR5, WDR5B, WDR69, ATG10, ATG3, ATG4A, ATG4B, ATG4C, ATG4D, ATG7, MOCS3, UBA1, UBA2, UBA6, GABARAP, GABARAPL1, GABARAPL2, GABARAPL3, LOC652191, MAP1LC3A, MAP1LC3B, MAP1LC3B2, MAP1LC3C, SSSCA1, ULK3, ATG2A, ATG2B, VPS13A, VPS13C, VPS13D, NLE1, WDR45, WDR45L, WIPI1, WIPI2, ABCB6, APEX1, ATG9A, ATG9B, C11orf24, C20orf151, C2orf16, CIC, FGFR1, FLG, FLG2, FMN2, FNDC1, IWS1, LEO1, MAP7D1, Mb3875, MLL2, MLL4, MUC1, NACA, PCLO, POLR2A, PRB3, REEP6, RERE, SETD5, ZNF828, CUX1, DNAH5, DNAH8, LOC652164, LOC653513, LOC727927, MY018A, PDE4DIP, TIAF1, SNX1, SNX18, SNX2, SNX3, SNX32, SNX5, SNX6, BCAS3, PRAF2, WDR45, WER45L, WIPI1, WIPI2, AZI1, CCDC88C, DCD42BPB, DSP, DSPP, HIP1, KIF16B, LAMA2, SPTBN5, SYNE2, TAX1BP1, UACA, ACTA1, ACTA2, ACTB, ACTC1, ACTG1, ACTG2, ACTR2, ACTR3, ACTR3B, ACTR3C, ENSP00000380341, SNX2, SNX30, SNX33, SNX4, SNX7, IGF2R, CLSTN1, HPVS34, PIK3C2A, PIK3C2B, PIK3C2G, PIK3C3, PIK3CA, PIK3CB, PIK3CD, PIK3CG, CUX1, DNAH5, DNAH8, LOC652164, LOC653513, LOC727927, MY018A, PDE4DIP, TIAF1, LOC100294331, SLC2A1, SLC2A14, SLC2A2, SLC2A3, SLC2A4, SLC2A5, SLC2A7, SLC2A9, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17UGT2B28, UGT2B4, UGT2B7, UGT3A2, UGT8, LOC652797, PKLR, PKM2, FLVCR1, FLVCR2, LOC100133772, SLC16A5, ANTXR1, ATM, ATR, GCN1L1, LOC440354, LOC648152, LOC651610, LOC651921, LOC731751, MMAB, MTOR, PRKDC, SERPINA2, SMG1, TRRAP, ANTXR1, ATM, ATR, LOC100288704, LOC440354, LOC648152, LOC651610, LOC651921, LOC731751, MMAB, PRKDC, SERPINA2, SMG1, TRRAP, DCD42BPA, EZR, UVRAG, WDR87, ARHGAP17, ARHGAP44, SH3GL1, SH3GL2, SH3GL3, SH3GLB1, SH3GLB2, DRAM, DRAM1, DRAM2, TMEM150A, TMEM150B, TMEM150C, AMBRA1, FBXW7, KATNB1, POC1A, POC1B, SPAG16, WDR38, WDR47, WDR5, WDR5B, WDR69, AKAP9, CGNL1, CIT, CLIP1, DHDDS, KIF15, MYH1, MYH13, MYH4, MYH8, RB1CC1, C120RF44, EEA1, FGD4, FGD5, FGD6, HGS, MTMR3, MTMR4, PIKFYVE, PLEKHF2, RUFY1, RUFY2, WDFY2, ZFYVE1, ZFYVE28, BAK1, BAX, BCL2, BCL2A1, BCL2L1, BCL2L2, BOK, LOC100289713, MCL1, DLG1, DLG2, DLG3, DLG4, GOPC, GRIP1, GRIP2, INADL, LGALS12, LIN7A, LIN7B, LIN7C, LLGL1, LNX1, MPDZ, MPP2, MPP3, MYH2, MYH3, MYH6, MYH7, NCOA2, PDZRN3, SNTA1, SNTB1, SNTB2, SNTG1, SNTG2, TMEM49, RAB11A, RAB11B, RAB17, RAB1A, RAB1B, RAB22A, RAB31, RAB43, RAB5A, RAB5B, RAB5C, RAB6A, IL18R1, IL1RAP, MYD88, TRAP, TLR1, TLR10, TLR2, TLR4, TLR6, RUBICON, P62/SQSTM1, C6orf106, HERC2, MIB1, MIB2, NBR1, SQSSTM1, ZZEF1, BIF-1, VMP1, TP531NP1, TP531NP2, BNIP3, BNIP3L, ARMC4, KPNA1, KPNA2, KPNA3, KPNA4, KPNA5, KPNA6, LC3B, GFAP, EF1A, TM9SF1, TMEM166, TMEM74, ULK3, P27, APE1, ARP2, and ARP3.

3. The method of claim 1, wherein the sample of gastrointestinal secretion is obtained from a colon of the warm-blooded animal.

4. The method of claim 2, wherein the gastrointestinal disorder is chronic diarrhea.

5. The method of claim 1, further comprising analyzing the sample of gastrointestinal secretion to determine whether a level of cytokeratin in the sample is abnormal, and correlating an abnormal level of cytokeratin in the sample with the gastrointestinal disorder in the warm-blooded animal from which the sample was obtained.

6. The method of claim 5, wherein the cytokeratin is selected from a subtype of cytokeratin I and subtype of cytokeratin II.

7. The method of claim 1, further comprising analyzing the sample of gastrointestinal secretion to determine whether levels of digestive enzymes, or antimicrobial proteins in the sample are abnormal, and correlating an abnormal level of digestive enzymes, or antimicrobial proteins in the sample with the gastrointestinal disorder in the warm-blooded animal from which the sample was obtained.

8. The method of claim 7, wherein the digestive enzymes are selected from amylase, lipase, and peptidase.

9. The method of claim 1, wherein the abnormal level comprises a raised level.

10. The method of claim 4, wherein the method further comprises treating the warm-blooded animal with chronic diarrhea by adjusting or regulating abnormal level of the autophagy-related protein in the warm-blooded animal with chronic diarrhea.

11. The method of claim 1, wherein the method further comprise comparing results obtained from an analysis performed on the sample of gastrointestinal secretions obtained from the warm-blooded animal with results obtained from analyses performed on a control group in order to determine the differences between each.

12. The method of claim 11, wherein the control group comprises warm-blooded animals diagnosed as having a healthy colon.

13. The method of claim 12, wherein the control group further comprises warm-blooded animals that have been diagnosed with chronic diarrhea or another gastrointestinal disorder.

14. A method for diagnosing chronic diarrhea in a warm-blooded animal comprising:
obtaining a sample of a gastrointestinal secretion from a colon of the warm-blooded animal;
analyzing the sample of gastrointestinal secretion to determine the sample's levels of a autophagy-related protein selected from BRSK1, BRSK2, CAMK1, CAMK1D, CAMK1G, CAMK2A, CAMK2B, CAMK2D, CAMK2G, DAPK3, DCLK1, MARK1, MARK2, MARK3, MARK4, NUAK1, NUAK2, PLK3, PLK4, PNCK, PRKAA1, PRKAA2, QSK, SIK1, SIK2, SNRK, STK33, STK36, ULK1, ULK2, ULK4, AKT1, AKT2, AKT3, ATG13, CHD9, HARBI1, KIAA0652, RPS6 KB1, RPS6 KB2, SGK1, SGK2, SGK3, CALCOCO1, CCDC21, MY018A, PHLDB2, PLCXD2, ROCK1, TMF1, TPR, BECN1, BECN111, CCDC88A, CEP12, CROCC, CTTNBP2, CTTNBP2NL, DNAH9, EPS15, KIF21A, KIF21B, MYH10, MYH11, MYH9, MY05C, NINL, RAB11FIP3, RAB11FIP4, ROCK2, SWAP70, ATG14, ATG14L, C9orf96, CAMK1, CDKL2, HVPS15, MAPK1, MAPK3, MARK1, MARK2, MARK3, MARK4, PHKG1, HIK3R4, PRKAA1, PRKAA2, QSK, SIK1, SIK2, SIK3, TMEM74, ATG5, OSGEP, OSGEP11, ATG7, MOCS3, UBA1, UBA2, UBA3, UBA6, ATG10, CNNM2, CNNM4, ATG12, DES, EEF1A1, EEF1A2, ENSP00000395046, GFAP, INA, LOC100293909, NEFL, NEFM, PRPH, PRPH2, VIM, ACTA1, ACTA2, ACTB, ACTBL2, ACTC1, ACTG1, ACTG2, ATG1611, ATG16L2, DCTN1, EPS1511, FBXW7, ITSN1, KIF5A, LOC653269, P704P, PAFAH1B1, SNRNP40, TM9SF1, TM9SF2, TM9SF3, TM9SF4, WDR5, WDR5B, WDR69, ATG10, ATG3, ATG4A, ATG4B, ATG4C, ATG4D, ATG7, MOCS3, UBA1, UBA2, UBA6, GABARAP, GABARAPL1, GABARAPL2, GABARAPL3, LOC652191, MAP1LC3A, MAP1LC3B, MAP1LC3B2, MAP1LC3C, SSSCA1, ULK3, ATG2A, ATG2B, VPS13A, VPS13C, VPS13D, NLE1, WDR45, WDR45L, WIPI1, WIPI2, ABCB6, APEX1, ATG9A, ATG9B, C11orf24, C20orf151, C2orf16, CIC, FGFR1, FLG, FLG2, FMN2, FNDC1, IWS1, LEO1, MAP7D1, Mb3875, MLL2, MLL4, MUC1, NACA, PCLO, POLR2A, PRB3, REEP6, RERE, SETD5, ZNF828, CUX1, DNAH5, DNAH8, LOC652164, LOC653513, LOC727927, MY018A, PDE4DIP, TIAF1, SNX1, SNX18, SNX2, SNX3, SNX32, SNX5, SNX6, BCAS3, PRAF2, WDR45, WER45L, WIPI1, WIPI2, AZI1, CCDC88C, DCD42BPB, DSP, DSPP, HIP1, KIF16B, LAMA2, SPTBN5, SYNE2, TAX1BP1, UACA, ACTA1, ACTA2, ACTB, ACTC1, ACTG1, ACTG2, ACTR2, ACTR3, ACTR3B, ACTR3C, ENSP00000380341, SNX2, SNX30, SNX33, SNX4, SNX7, IGF2R, CLSTN1, HPVS34, PIK3C2A, PIK3C2B, PIK3C2G, PIK3C3, PIK3CA, PIK3CB, PIK3CD, PIK3CG, CUX1, DNAH5, DNAH8, LOC652164, LOC653513, LOC727927, MY018A, PDE4DIP, TIAF1, LOC100294331, SLC2A1, SLC2A14, SLC2A2, SLC2A3, SLC2A4, SLC2A5, SLC2A7, SLC2A9, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17UGT2B28, UGT2B4, UGT2B7, UGT3A2, UGT8, LOC652797, PKLR, PKM2, FLVCR1, FLVCR2, LOC100133772, SLC16A5, ANTXR1, ATM, ATR, GCN1L1, LOC440354, LOC648152, LOC651610, LOC651921, LOC731751, MMAB, MTOR, PRKDC, SERPINA2, SMG1, TRRAP, ANTXR1, ATM, ATR, LOC100288704, LOC440354, LOC648152, LOC651610, LOC651921, LOC731751, MMAB, PRKDC, SERPINA2, SMG1, TRRAP, DCD42BPA, EZR, UVRAG, WDR87, ARHGAP17, ARHGAP44, SH3GL1, SH3GL2, SH3GL3, SH3GLB1, SH3GLB2, DRAM, DRAM1, DRAM2, TMEM150A, TMEM150B, TMEM150C, AMBRA1, FBXW7, KATNB1, POC1A, POC1B, SPAG16, WDR38, WDR47, WDR5, WDR5B, WDR69, AKAP9, CGNL1, CIT, CLIP1, DHDDS, KIF15, MYH1, MYH13, MYH4, MYH8, RB1CC1, C120RF44, EEA1, FGD4, FGD5, FGD6, HGS, MTMR3, MTMR4, PIKFYVE, PLEKHF2, RUFY1, RUFY2, WDFY2, ZFYVE1, ZFYVE28, BAK1, BAX, BCL2, BCL2A1, BCL2L1, BCL2L2, BOK, LOC100289713, MCL1, DLG1, DLG2, DLG3, DLG4, GOPC, GRIP1, GRIP2, INADL, LGALS12, LIN7A, LIN7B, LIN7C, LLGL1, LNX1, MPDZ, MPP2, MPP3, MYH2, MYH3, MYH6, MYH7, NCOA2, PDZRN3, SNTA1, SNTB1, SNTB2, SNTG1, SNTG2, TMEM49, RAB11A, RAB11B, RAB17, RAB1A, RAB1B, RAB22A, RAB31, RAB43, RAB5A, RAB5B, RAB5C, RAB6A, IL18R1, IL1RAP, MYD88, TIRAP, TLR1, TLR10, TLR2, TLR4, TLR6, RUBICON, P62/SQSTM1, C6orf106, HERC2, MIB1, MIB2, NBR1, SQSSTM1, ZZEF1, BIF-1, VMP1, TP531NP1, TP531NP2, BNIP3, BNIP3L, ARMC4, KPNA1, KPNA2, KPNA3, KPNA4, KPNA5, KPNA6, LC3B, GFAP, EF1A, TM9SF1, TMEM166, TMEM74, ULK3, P27, APE1, ARP2, and ARP3;
comparing results obtained from the analysis of the sample with results obtained from a control group in order to determine if the levels of the autophagy-related protein in the sample are abnormal; and
correlating an abnormal level of the autophagy-related protein in the sample with chronic diarrhea in the warm-blooded animal from which the sample was obtained.

15. The method of claim 14, further comprising analyzing the sample of gastrointestinal secretion to determine whether a level of cytokeratin in the sample is abnormal, and correlating an abnormal level of cytokeratin in the sample with the gastrointestinal disorder in the warm-blooded animal from which the sample was obtained.

16. The method of claim 15, wherein the cytokeratin is selected from CK1, CK2, CK5, CK6, CK9, CK10, CK14, CK16, and combinations thereof.

17. The method of claim 14, further comprising analyzing the sample of gastrointestinal secretion to determine whether levels of digestive enzymes, or antimicrobial proteins in the sample are abnormal, and correlating an abnormal level of digestive enzymes, or antimicrobial proteins in the sample with the gastrointestinal disorder in the warm-blooded animal from which the sample was obtained.

18. The method of claim 13, wherein the method further comprises treating the warm-blooded animal with chronic diarrhea by adjusting or regulating the abnormal level of the autophagy-related protein.

19. A method for diagnosing chronic diarrhea in a warm-blooded animal comprising:
obtaining a sample of a gastrointestinal secretion from a colon of the warm-blooded animal;
analyzing the sample of gastrointestinal secretion to determine the sample's levels of a autophagy-related protein selected from BRSK1, BRSK2, CAMK1, CAMK1D, CAMK1G, CAMK2A, CAMK2B, CAMK2D, CAMK2G, DAPK3, DCLK1, MARK1, MARK2, MARK3, MARK4, NUAK1, NUAK2, PLK3, PLK4, PNCK, PRKAA1, PRKAA2, QSK, SIK1, SIK2, SNRK, STK33, STK36, ULK1, ULK2, ULK4, AKT1, AKT2, AKT3, ATG13, CHD9, HARBI1, KIAA0652, RPS6 KB1, RPS6 KB2, SGK1, SGK2, SGK3, CALCOCO1, CCDC21, MY018A, PHLDB2, PLCXD2, ROCK1, TMF1, TPR, BECN1, BECN111, CCDC88A, CEP12, CROCC, CTTNBP2, CTTNBP2NL, DNAH9, EPS15, KIF21A, KIF21B, MYH10, MYH11, MYH9, MY05C, NINL, RAB11FIP3, RAB11FIP4, ROCK2, SWAP70, ATG14, ATG14L, C9orf96, CAMK1, CDKL2, HVPS15, MAPK1, MAPK3, MARK1, MARK2, MARK3, MARK4, PHKG1, HIK3R4, PRKAA1, PRKAA2, QSK, SIK1, SIK2, SIK3, TMEM74, ATG5, OSGEP, OSGEP11, ATG7, MOCS3, UBA1, UBA2, UBA3, UBA6, ATG10, CNNM2, CNNM4, ATG12, DES, EEF1A1, EEF1A2, ENSP00000395046, GFAP, INA, LOC100293909, NEFL, NEFM, PRPH, PRPH2, VIM, ACTA1, ACTA2, ACTB, ACTBL2, ACTC1, ACTG1, ACTG2, ATG1611, ATG16L2, DCTN1, EPS1511, FBXW7, ITSN1, KIF5A, LOC653269, P704P, PAFAH1B1, SNRNP40, TM9SF1, TM9SF2, TM9SF3, TM9SF4, WDR5, WDR5B, WDR69, ATG10, ATG3, ATG4A, ATG4B, ATG4C, ATG4D, ATG7, MOCS3, UBA1, UBA2, UBA6, GABARAP, GABARAPL1, GABARAPL2, GABARAPL3, LOC652191, MAP1LC3A, MAP1LC3B, MAP1LC3B2, MAP1LC3C, SSSCA1, ULK3, ATG2A, ATG2B, VPS13A, VPS13C, VPS13D, NLE1, WDR45, WDR45L, WIPI1, WIPI2, ABCB6, APEX1, ATG9A, ATG9B, C11orf24, C20orf151, C2orf16, CIC, FGFR1, FLG, FLG2, FMN2, FNDC1, IWS1, LEO1, MAP7D1, Mb3875, MLL2, MLL4, MUC1, NACA, PCLO, POLR2A, PRB3, REEP6, RERE, SETD5, ZNF828, CUX1, DNAH5, DNAH8, LOC652164, LOC653513, LOC727927, MY018A, PDE4DIP, TIAF1, SNX1, SNX18, SNX2, SNX3, SNX32, SNX5, SNX6, BCAS3, PRAF2, WDR45, WER45L, WIPI1, WIPI2, AZI1, CCDC88C, DCD42BPB, DSP, DSPP, HIP1, KIF16B, LAMA2, SPTBN5, SYNE2, TAX1BP1, UACA, ACTA1, ACTA2, ACTB, ACTC1, ACTG1, ACTG2, ACTR2, ACTR3, ACTR3B, ACTR3C, ENSP00000380341, SNX2, SNX30, SNX33, SNX4, SNX7, IGF2R, CLSTN1, HPVS34, PIK3C2A, PIK3C2B, PIK3C2G, PIK3C3, PIK3CA, PIK3CB, PIK3CD, PIK3CG, CUX1, DNAH5, DNAH8, LOC652164, LOC653513, LOC727927, MY018A, PDE4DIP, TIAF1, LOC100294331, SLC2A1, SLC2A14, SLC2A2, SLC2A3, SLC2A4, SLC2A5, SLC2A7, SLC2A9, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17UGT2B28, UGT2B4, UGT2B7, UGT3A2, UGT8, LOC652797, PKLR, PKM2, FLVCR1, FLVCR2, LOC100133772, SLC16A5, ANTXR1, ATM, ATR, GCN1L1, LOC440354, LOC648152, LOC651610, LOC651921, LOC731751, MMAB, MTOR, PRKDC, SERPINA2, SMG1, TRRAP, ANTXR1, ATM, ATR, LOC100288704, LOC440354, LOC648152, LOC651610, LOC651921, LOC731751, MMAB, PRKDC, SERPINA2, SMG1, TRRAP, DCD42BPA, EZR, UVRAG, WDR87, ARHGAP17, ARHGAP44, SH3GL1, SH3GL2, SH3GL3, SH3GLB1, SH3GLB2, DRAM, DRAM1, DRAM2, TMEM150A, TMEM150B, TMEM150C, AMBRA1, FBXW7, KATNB1, POC1A, POC1B, SPAG16, WDR38, WDR47, WDR5, WDR5B, WDR69, AKAP9, CGNL1, CIT, CLIP1, DHDDS, KIF15, MYH1, MYH13, MYH4, MYH8, RB1CC1, C120RF44, EEA1, FGD4, FGD5, FGD6, HGS, MTMR3, MTMR4, PIKFYVE, PLEKHF2, RUFY1, RUFY2, WDFY2, ZFYVE1, ZFYVE28, BAK1, BAX, BCL2, BCL2A1, BCL2L1, BCL2L2, BOK, LOC100289713, MCL1, DLG1, DLG2, DLG3, DLG4, GOPC, GRIP1, GRIP2, INADL, LGALS12, LIN7A, LIN7B, LIN7C, LLGL1, LNX1, MPDZ, MPP2, MPP3, MYH2, MYH3, MYH6, MYH7, NCOA2, PDZRN3, SNTA1, SNTB1, SNTB2, SNTG1, SNTG2, TMEM49, RAB11A, RAB11B, RAB17, RAB1A, RAB1B, RAB22A, RAB31, RAB43, RAB5A, RAB5B, RAB5C, RAB6A, IL18R1, IL1RAP, MYD88, TIRAP, TLR1, TLR10, TLR2, TLR4, TLR6, RUBICON, P62/SQSTM1, C6orf106, HERC2, MIB1, MIB2, NBR1, SQSSTM1, ZZEF1, BIF-1, VMP1, TP531NP1, TP531NP2, BNIP3, BNIP3L, ARMC4, KPNA1, KPNA2, KPNA3, KPNA4, KPNA5, KPNA6, LC3B, GFAP, EF1A, TM9SF1, TMEM166, TMEM74, ULK3, P27, APE1, ARP2, and ARP3;
comparing results obtained from the analysis of the sample with results obtained from a control group in order to determine if the levels of the autophagy-related protein in the sample are abnormal;
correlating an abnormal level of the autophagy-related protein in the sample with chronic diarrhea in the warm-blooded animal from which the sample was obtained; and
treating the warm-blooded animal with chronic diarrhea by adjusting or regulating the abnormal level of the autophagy-related protein.

20. The method of claim 19, further comprising analyzing the sample of gastrointestinal secretion to determine whether a level of cytokeratin in the sample is abnormal, and correlating an abnormal level of cytokeratin in the sample with the gastrointestinal disorder in the warm-blooded animal from which the sample was obtained.
